(19) United States
(12) Patent Application Publication
(10) Pub. No.: US 2009/0099503 A1
(43) Pub. Date: Apr. 16, 2009

(54) Ozone treatment apparatus for acne

(76) Inventor: Kunihiko Mitsuda, Tokyo (JP)

Correspondence Address: Edwards Angell Palmer & Dodge LLP, P.O. Box 55874, Boston, MA, 02205, US

(21) Appl. No.: 12/001,334

(22) Filed: Dec. 11, 2007

(30) Foreign Application Priority Data
Oct. 15, 2007 (JP) 2007-267706

(51) Int. Cl. A61M 37/00

(52) U.S. Cl. 604/25

(57) ABSTRACT

An ozone treatment apparatus for acne of the present invention comprises: an ozone gas or ozone water generator; a skin coupling device, which is placed in contact with the skin, and has pores for spraying the ozone gas or ozone water generated into a diseased part; and an aspirator, which forms a vacuum inside of the skin coupling device placed in contact with the skin, impregnates the injected ozone gas or ozone water into the skin, and suctions the ozone gas or ozone water from the skin coupling device. It enables the ozone gas or ozone water to enter the deep part of pores, and to apply a sufficient sterilization effect to bacteria in the deep part.

### BACKGROUND OF THE INVENTION

The present invention relates to a treatment apparatus, which, for the purpose of treating various human skin problems, is placed in contact with the skin to impregnate ozone gas or ozone water into the surface of the skin or inside the skin. In particular, it relates to an ozone treatment apparatus to be used for acne treatment.

In recent years, attention has been given to equipment applications using the high bactericidal action of ozone (O₃) in a range of fields.

At present, the biggest use of ozone is for the purification of water supplies, where it is used for the purposes of sterilization and deodorization of water. In addition to this, ozone is used for (i) sterilization of food and maintenance of freshness, (ii) sterilization and reduction of organic substances in water in swimming pools and baths, (iii) sterilization, decolorization and organic substance reduction of water for use in aquariums, fish farms and zoos, (iv) sterilization of greenhouse cultures and water cultures, (v) sterilization and deodorization of the inside of refrigerators and freezing rooms, (vi) decolorization of pulp, (vii) sterilization and deodorization of room air, (viii) deodorization and toxic substance removal of industrial liquid waste, (ix) removal of trace organic substances in semiconductors, and the like.

Furthermore, like other bactericides and oxidizing agents, since ozone does not have residual properties, but does have a function of dissolving harmful organic substances and making them harmless, its range of employment is expected to expand rapidly in the future due to the recent global movement to attach more importance to the environment.

Moreover, attempts to use ozone in medical treatment have been made from the middle of the 20^{th} century, and have attracted wide public attention in applications for the sterilization and deodorization of patients' rooms in hospitals, for the sterilization of medical equipment, and for medical treatment and the like. The device in which ozone is most used in such medical fields is an ozonator for dental caries treatment (http://jp.kavo.com/produkte/therapei#insstrmente/healozone/healozone.asp?navid=26&1a n=Js).

Ozone has three oxygen atoms, which is a very unstable state, and has a characteristic that it tries to become O₂, which is a stable oxygen molecule, by transferring one of its atoms to another substance. Accordingly it has a strong oxygen behavior. The strength of its oxygen behavior is second to fluorine, and it has stronger oxidizability than hydrogen peroxide, chlorine, or hypochlorous acid, so most organic substances and metals can be oxidized by ozone.

Ozone for medical applications is used as an ozone containing gas (ozone gas), or ozone water in which ozone is dissolved in water. However, in most cases, it is used for (i) sterilization, (ii) deodorization, (iii) removal of organic substances, (iv) decolorization, (v) removal of toxic substances, (vi) synthesis of chemical substances, and the like.

The normal dermal inhabitant that mostly causes the skin disorder dermatosis is Staphylococcus epidermidis, which is a type of staphylococcus. It can reach hundreds to hundreds of thousands per 1 cm² of skin surface. Propionibacterium acnes follow, and there are tens to millions per 1 cm² of acne. Staphylococcus epidermidis and propionibacterium acnes both have an affinity for sebum cutaneum. Furthermore, since propionibacterium acnes involve so-called anaerobic bacteria, which dislike air, then as pore blocking progresses, they increase in a human follicle. If sebum cutaneum stored in the follicle is broken down by the bacteria, it changes to free fatty acid, and the free fatty acid becomes a phlogogenic material, which irritates skin cells strongly. In this manner, the aforementioned propionibacterium acnes involve the progress of conditions from a state that is called comedo, in which sebum cutaneum fills the follicle, to acne. Eventually, the condition of inflammation becomes papuloerythematous, and if the papule part suppurates, a pustule is formed.

For acne treatment devices, currently there are a method (abbreviated name PDP) (http://www.nikibi.1.net/30/post#24.html) in which propionibacterium acnes are destroyed by radiating a light in the ultraviolet range (from 400 nm to 500 nm) on the skin to generate singlet oxygen (¹O₂) after applying aminolevulinic acid onto a skin surface, and a method such as photo pneumatic therapy (abbreviated name PPx) (http://www.medieth.com/camp/cam#nikibi.html) in which propionibacterium acnes are destroyed by radiating a flash of light of 440 nm to 550 nm while suctioning the skin surface.

However, there are problems in that since most of these types of acne treatment device used at present are ones using light, treatment sometimes cannot be applied because of the skin color or immediately after getting a tan, and since light does not cause damage directly to propionibacterium acnes, the treatment effect does not appear immediately, hence several further cycles of treatment are required.

Furthermore, existing acne treatment devices only spray ozone gas or ozone water on the surface of the diseased skin, so the toxic effect of the sterilization is lost immediately due to the instability of ozone gas. Therefore, it is very difficult to permeate (impregnate) ozone gas or the like into the skin pores or deeper. Moreover, when the disease is deep inside of glandula sebacea, as is the case with acne, there is a problem in that sufficient ozone gas or ozone water does not reach the diseased part.

### SUMMARY OF THE INVENTION

Therefore, the present invention has an object of treating skin disorders caused by bacteria; by creating negative pressure on the surface of the skin to extract sebum cutaneum in glandula sebacea in the skin surface, and permeate ozone gas or ozone water deep into the skin, and by using injector needles in order to inject the ozone gas or the like more completely into the skin.

The ozone treatment apparatus for acne of the present invention comprises: an ozone gas or ozone water generator, in which there is provided a valve that turns the injection of ozone gas or ozone water on and off; a skin coupling device, which is placed in contact with the skin such that ozone gas or ozone water does not leak outside of the skin, has pores for spraying ozone gas or ozone water into a diseased part, and has at least one or more injection needles for injecting ozone gas or ozone water into the skin; an aspirator, which forms a negative pressure state inside of the coupling device that is placed in close contact with the skin, extracts sebum cutaneum and dirt from the cutaneous gland, impregnates ozone gas or ozone water deep into the glandula sebacea and follicle, and also suctions the ozone gas or ozone water; an ozone gas or ozone water decomposition device, which decomposes the ozone gas or ozone water suctioned by the aspirator; and a pressure device, which pressurizes the skin to impregnate the ozone gas or ozone water into the glandula sebacea and follicle, and has a valve for turning the pressure on and off.

By spraying and injecting ozone gas or ozone water into subcutaneous tissue having a diseased part (acne), which is suctioned by vacuum, it is possible for the ozone gas or ozone water to enter into the deepest part of the pore, and apply a sufficient sterilization effect to bacteria in the glandula sebacea involved and the deepest part of the follicle.

### BRIEF DESCRIPTION OF THE DRAWINGS

FIG. 1 is a conceptual diagram of an embodiment of an ozone treatment apparatus for acne of the present invention.

FIG. 2 shows a detachable adaptor, which is to be mounted on the end of a skin coupling device of the ozone treatment apparatus of the present invention as shown in FIG. 1. FIG. 2A shows a plan view thereof, and FIG. 2B shows a diagram showing the cross-section indicated by arrows A-A in FIG. 2A.

FIG. 3 shows an operation procedure of the ozone treatment apparatus of the present invention. FIG. 3A, FIG. 3B, FIG. 3C and FIG. 3D show respectively: a state in which an ozone valve and a vacuum valve are opened and ozone gas (water) is drawn into the skin coupling device; a state in which the ozone valve and a compressor valve are closed, the vacuum valve is opened, and the skin is suctioned; a state in which the ozone valve is opened to normalize the pressure inside of the coupling device (atmospheric pressure), and ozone gas (water) is sprayed into the skin; and a state in which the compressor valve is opened, the ozone valve and the vacuum valve are closed, and the skin is pressurized (however, the detachable coupling is not shown in the figure here).

FIG. 4 shows a state in which the detachable adaptor is mounted on the skin coupling device, and is put in contact with the diseased part of the skin (acne). FIG. 4A, FIG. 4B, FIG. 4C and FIG. 4D show states corresponding to FIG. 3A, FIG. 3B, FIG. 3C and FIG. 3D, respectively.

FIG. 5 shows a perspective view in which the detachable adaptor is removed from the skin coupling device.

FIG. 6 shows a state in which the ozone treatment apparatus of the present invention is placed in contact with the diseased part (acne) to treat it.

### DETAILED DESCRIPTION OF THE PREFERRED EMBODIMENTS

Hereunder is a detailed description of an embodiment of an ozone treatment apparatus for acne of the present invention with reference to the appended drawings.

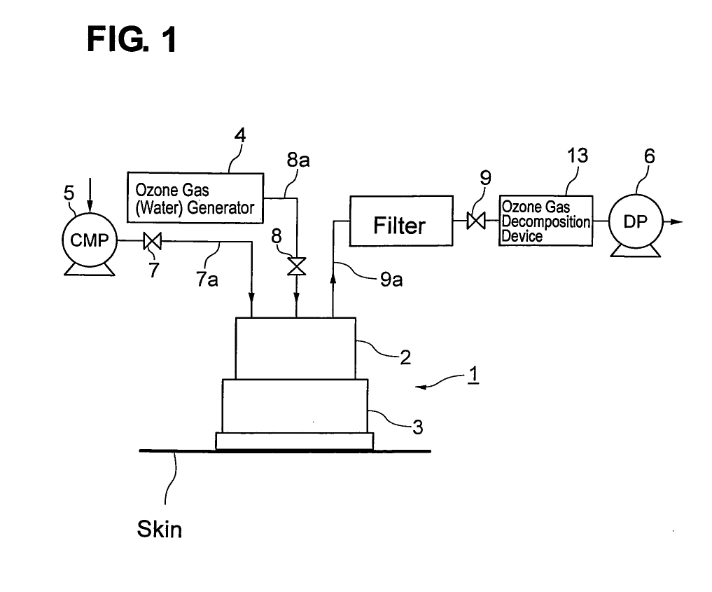

FIG. 1 shows an embodiment of the ozone treatment apparatus for acne 1 of the present invention. It comprises: an ozone gas or ozone water generator 4 (referred to hereunder as “ozone gas (water) generator”); a skin coupling device 2, which is placed in contact with the skin such that ozone gas or ozone water does not leak outside the skin, has pores for spraying ozone gas or ozone water into a diseased part, and has at least one or more injection needles 10 for injecting ozone gas or ozone water into the skin; an injection device having a valve 8, which injects the ozone gas or ozone water between the skin coupling device 2 and the skin, and turns the injection on and off; a pressure device 5 (compressor) having a valve 7 (compressor valve), which pressurizes the skin to impregnate the ozone gas or ozone water into the skin, and turns the pressure on and off; an aspirator 6 (vacuum pump), which suctions the ozone gas or ozone water injected from the coupling device 2 placed in contact with the skin via the valve 7 (vacuum pump valve); and an ozone gas decomposition device 13, which decomposes the suctioned ozone gas or ozone water.

Here, it is considered that ozone gas has several tens of times the sterilization effect of ozone water (Yoshikatsu Kashiwagi et al, Study Regarding Difference in Sterilization Effect Between Ozone Gas and Ozone Water, Medical Instrumentation Vol. 57, Issue 4 (1987, refer to page 184), Yasuo Ito, Wonders of Ozone, Kodansha Blueback, pages 87 to 95).

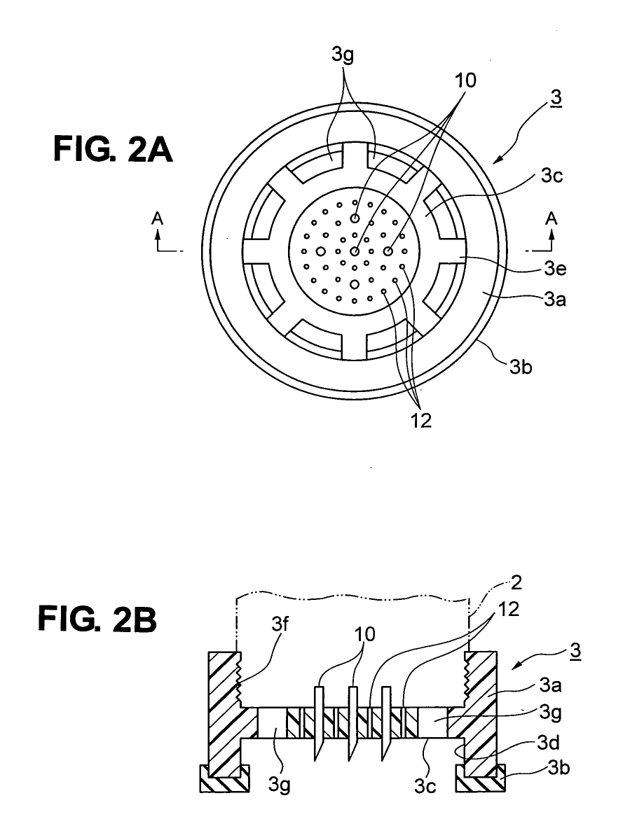

In particular, as shown in FIG. 2B, a detachable adaptor 3 to be installed at the end of the skin coupling device 2 such that it can be attached or removed freely is made from a plastic material (for example, acrylic resin (PMMA)). It comprises a joining part, which connects an annular main body part 3a and its inner side annular wall 3d via leg parts 3e, a contact part 3b made from silicon rubber, which is installed on the bottom portion of the main body part 3a, and a female screw part 3f, which is screwed onto a male screw part at the end of the coupling device 2. As shown in FIG. 2B, a plurality of, for example five, injection needles 10, whose tips are sharp, is embedded in the joining part 3c so as to protrude from the top and bottom surfaces of the joining part 3c. Furthermore, many pores 12 (hole diameter: 100 μm) are formed concentrically with the injection needles 10 provided in the center of the joining part 3c.

Here, the injection needles 10 used have hollow bodies, and their tips are sharpened. For example, their lengths are 10 to 20 mm, the diameter of the hollow body (hole) is 80 μm, and the outer diameter is 200 μm (as commercial products, painless needles made by Okano Kogyo Co. Ltd. are suitable, trade name: Nanopass 33).

Furthermore, for the material of the injection needles 10, aluminum, stainless steel, tantalum, or molybdenum is suitable.

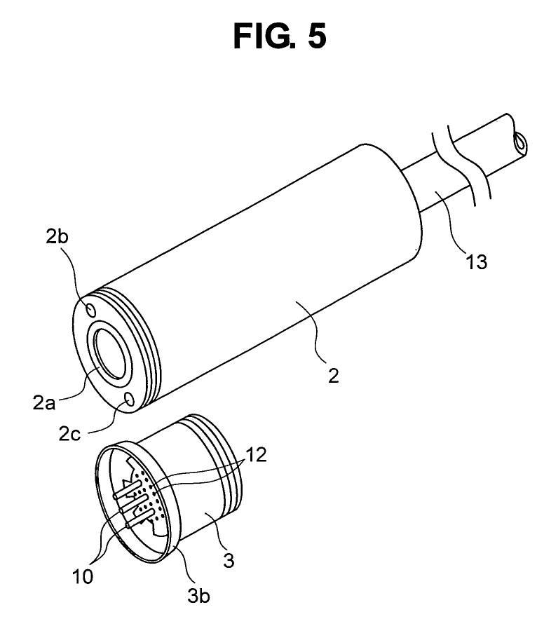

The skin coupling device 2 uses stainless steel, titanium or the like. For example, the diameter is 20 to 30 mm, and the length is 100 to 150 mm. An example is shown in FIG. 5.

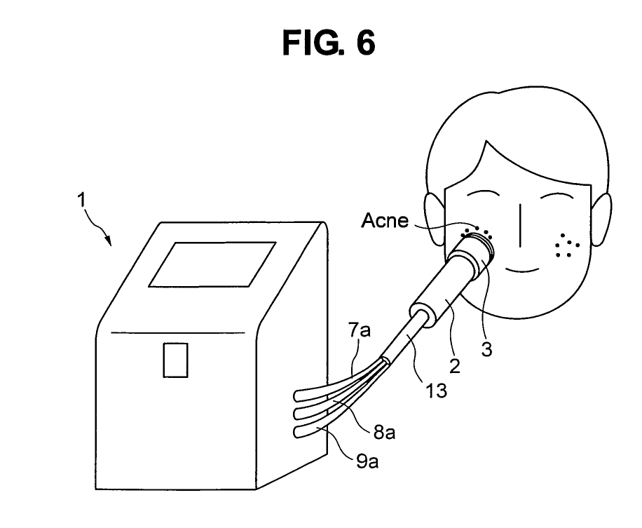

As shown in FIG. 6, it is also possible to store the ozone gas (water) generator, the compressor, the vacuum pump, the ozone gas decomposition device, and the like in a casing, and extend the skin coupling device from the casing for use as a portable treatment apparatus.

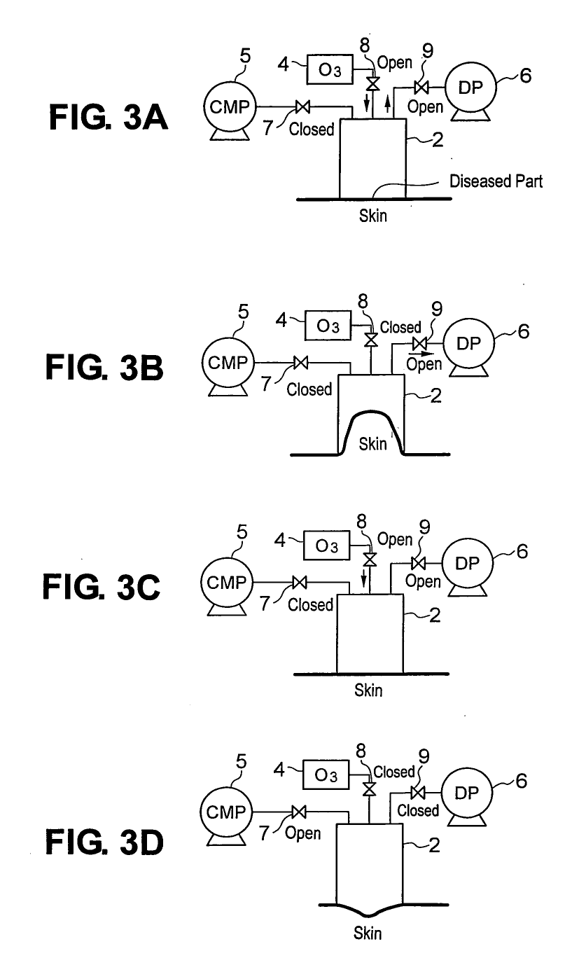

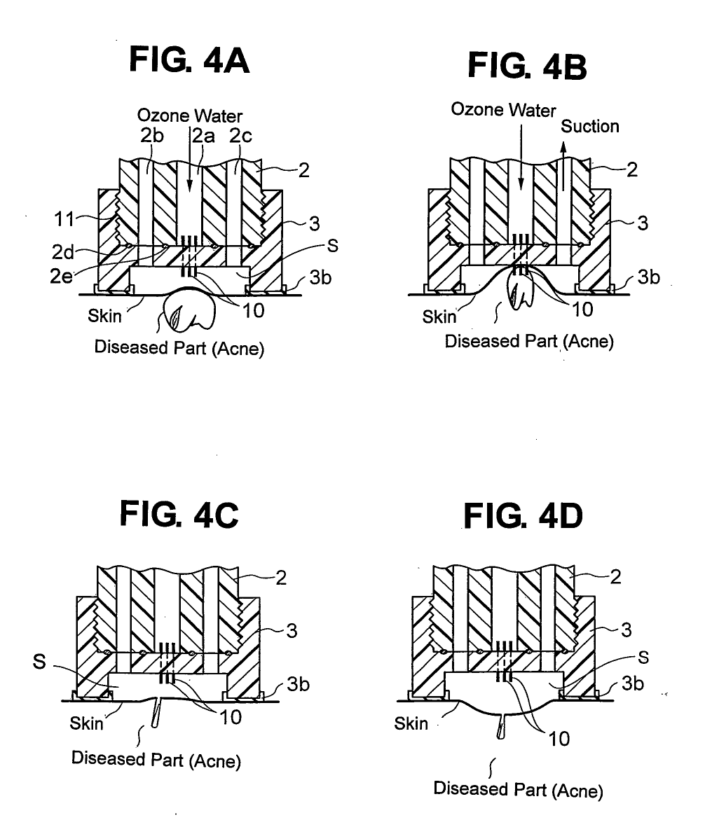

Next is a description of an operating method of the ozone treatment apparatus for skin disorder 1 using FIG. 3 and FIG. 4.

Firstly, as shown in FIG. 3A, FIG. 4A and FIG. 6, the detachable adaptor 3 mounted on the skin coupling device 2 is pressed in contact with the skin such that it encloses the diseased part of the skin (acne), the ozone valve 8 and the vacuum valve 9 are opened, and ozone gas (water) is drawn into a central cavity 2a of the coupling device 2 from an ozone gas (water) generator 4 via a tube 8a and the ozone valve 8 (first step). For the detachable adaptor 3, as alternatives to one in which the injection needles 10 are implanted and many pores 12 are formed, it is also possible to prepare one in which only injection needles 19 are embedded, or one in which only pores 12 are formed, and attach them selectively depending on the degree and condition of the skin disorder such as acne. Here, two O-rings 2d and 2e intervene between the contacting surfaces of the end of the skin coupling device 2 and the detachable adaptor 3.

Next, as shown in FIG. 3B and FIG. 4B, the ozone valve 8 and the compressor valve 7 are closed, and the part of the skin enclosed by the coupling device 2 is suctioned using the vacuum valve 9 of the vacuum pump 6 at a vacuum pressure of 1 to 6 kPa through a tube 9a and a hole 2c. In this manner, the diseased part (acne) is pulled up and makes contact with the injection needles 10 embedded in the adaptor 3 as in FIG. 4B, so that the injection needles 10 are inserted into the skin (step 2).

Then, as shown in FIG. 3C and FIG. 4C, the ozone valve 8 is opened, normalizing the pressure (atmospheric pressure) in the inside of the coupling device 2, and ozone gas (water) is injected into the skin through the holes of the injection needles 10, and sprayed into a space S via holes 3g formed between the pores 12 and the leg parts 3e of the adaptor 3 in contact (third step).

Finally, as shown in FIG. 3D and FIG. 4D, the compressor valve 7 is opened, the ozone valve 8 and the vacuum valve 9 are closed, and then the skin is pressurized at a pressure of 1 to 3 kPa via the tube 7a and the hole 2c, and the ozone gas or ozone water is impregnated (permeated) into the skin (fourth step).

After repeating from the first step to the fourth step described above a predetermined number of times (several times), all the valves are closed, and the treatment of the diseased part (acne) is terminated.

In addition, by mixing vitamins or other medicines into the ozone gas or ozone water, the effect of the acne treatment can be further enhanced.

It is possible to use an ozone treatment apparatus of the present invention for the treatment of other skin disorders such as bedsores and the like, in addition to the treatment of acne.

1. An ozone treatment apparatus for acne comprising:
(a) an ozone gas or ozone water generator;
(b) skin coupling device, adapted and configured for placement in contact with skin, for injecting ozone gas or ozone water from the generator, and for spraying the ozone gas or ozone water onto or injecting the ozone gas or water into a diseased part of the skin;
(c) an aspirator, which is adapted and configured to suction the ozone gas or ozone water from said skin coupling device, and to form a vacuum inside of said skin coupling device; and
(d) a pressure device, which is adapted and configured to pressurize skin in the region of the skin coupling device to impregnate the ozone gas or ozone water into the skin; and
(e) a detachable adapter, adapted and configured for mounting to an end of the skin coupling device, the adapter being provided with one or more of a pore and an injection needle facilitating penetration of the ozone gas or ozone water into the skin for treatment of acne.

2. (canceled)

3. An ozone treatment apparatus according to claim 17, wherein at least one or more pores is provided in said skin coupling device in order to spray ozone gas or ozone water into the diseased part.

4. An ozone treatment apparatus according to claim 17, wherein a detachable adaptor is mounted onto an end of said skin coupling device, and said adaptor is provided with pores.

5. An ozone treatment apparatus according to claim 17, wherein a detachable adaptor is mounted onto an end of said skin coupling device, and said adaptor is provided with injection needles.

6. (canceled)

7. An ozone treatment apparatus according to claim 1, wherein an ozone gas or ozone water decomposition device is provided upstream of said aspirator.

8. An ozone treatment apparatus according to claim 1, wherein said pressure device is provided upstream of said skin coupling device.

9. A method of ozone treatment for acne comprising the steps of:
(a) providing an ozone treatment apparatus including a skin coupling device;
(b) placing the skin coupling device in contact with a region of skin of the patient to define a central cavity within which treatment will occur;
(c) flushing the central cavity with an ozone fluid;
(d) applying a vacuum to the central cavity, causing displacement of the region of skin toward a proximal end of the skin coupling device;
(e) equalizing pressure within the central cavity by allowing the ozone fluid to flow into the central cavity;
(f) pressurizing, with positive pressure, the central cavity to assist permeation of the ozone fluid into at least a portion of the region of skin.

10. The method of claim 10, further comprising mounting a detachable adapter to the skin coupling device.

11. The method of claim 11, further comprising the step of selecting the adapted based on the treatment features thereof.

12. The method of claim 10, wherein the displacement of the region of skin toward a proximal end of the skin coupling device causes the skin to contact at least one injection needle held within the skin coupling device, the contact allowing for injection of the ozone fluid into at least a portion of the region of skin.

13. The method of claim 12, wherein in the equalizing step, ozone fluid is injected through at least one injection needle.

14. The method of claim 10, wherein in the equalizing step, ozone fluid is sprayed onto at least a portion of the region of skin.

15. The method of claim 10, wherein the positive pressure applied is between about 1.0 and 3.0 kPa.

16. The method of claim 10, wherein the method further includes repeating the flushing, applying, equalizing and pressurizing steps for a predetermined number of times during one treatment.

17. An ozone treatment apparatus for acne comprising:
(a) a generator for generating ozone gas or ozone water;
(b) a skin coupling device, adapted and configured for placement in contact with a region of the skin of a patient, the skin coupling device being in fluid communication with the generator, and adapted and configured to receive ozone gas or ozone water from the generator, and for spraying the ozone gas or ozone water onto or injecting the ozone gas or water into a diseased part of said region of skin;
(c) an aspirator in fluid communication with the skin coupling device, the aspirator being adapted and configured to suction the ozone gas or ozone water from said skin coupling device, and being adapted and configured to form a vacuum inside of said skin coupling device to exert a negative pressure on said region of skin;
(d) a pressure device, which is adapted and configured to pressurize skin in said region of skin to impregnate the ozone gas or ozone water into said region of skin; and
(e) a detachable adapter, adapted and configured for mounting to an end of the skin coupling device, the adapter being provided with one or more of a pore and an injection needle facilitating penetration of the ozone gas or ozone water into the skin for treatment of acne.
